(19) United States
(12) Patent Application Publication
(10) Pub. No.: US 2011/0077568 A1
(43) Pub. Date: Mar. 31, 2011

(54) SPHERICAL SECTION HAND SPLINT

(76) Inventor: Charles L. Brown, Prior Lake, MN (US)

(21) Appl. No.: 12/894,594

(22) Filed: Sep. 30, 2010

Related U.S. Application Data
(60) Provisional application No. 61/247,333, filed on Sep. 30, 2009.

(51) Int. Cl. A61F 5/04

(52) U.S. Cl. 602/21; 602/22

(57) ABSTRACT

A flexible, low profile hand splint includes a section of a hollow sphere or ball that comfortably puts the hand in an at rest natural position, while immobilizing one or more anatomical hand parts with extended spherical sections thereby immobilizing fingers, knuckles and tendons. Straps hold the splint onto the palm and immobilize one or more of the fingers. An inverse version of the splint provides protection to the injured soft tissue of a palm.

### CROSS-REFERENCE TO RELATED APPLICATION

The present application is based on and claims the benefit of U.S. provisional patent application Ser. No. 61/427,333, filed Sep. 30, 2009.

### BACKGROUND OF THE INVENTION

This invention relates to a hand splint for initial injury care and to support healing of an injury and more particularly to a spherical section hand splint that puts all or part of the hand into a natural hand at rest position to immobilize injured anatomical parts of the hand.

The anatomical parts of the hand are subject to many different types of injuries, not limited to, but including, finger fractures, finger joint dislocations, tendon dislocations, finger and knuckle joint injuries, finger crush injuries, finger soft tissue injuries, incomplete tendon lacerations, sprains from jammed fingers, finger alignment injuries, finger and knuckle pain control issues, tendon repairs, laceration repairs, skin graphs, soft tissue injuries and acutely inflamed arthritic joints.

FIG. 1 shows a common prior art knuckle wrist immobilizing rigid splint or brace 10 which has a rigid aluminum brace 12 sewn in to immobilize the wrist 4 while an aluminum finger splint 14 extends therefrom for immobilized support of the finger 6. Rigid splint 10 may be used in treating carpal tunnel syndrome. This prior art splint 10 is cumbersome to put on and take off and offers poor hand mobility beyond the necessary immobilization of the injured anatomical part of the hand 2. With this type of rigid splint 10, the wearer has great difficulty to dress, drive a car, operate a computer, hold food and eating utensils and write with a pen or pencil.

Referring to FIG. 2, the hand 2 is shown with an injury in the palm 8 covered by a thick gauze bandage 16. Here the palm is typically not immobilized and the injured palm area 8 underneath the bandage 16 is subject to additional tenderness and pain due to lack of immobilization and protection.

There is a need for a splint that puts all or part of the hand 2 into a natural hand at rest position as shown in FIGS. 3 and 4 as resting on a ball or sphere 18 while immobilizing the injured anatomical parts of the hand, such as the knuckles and/or joints.

### SUMMARY OF THE INVENTION

A flexible, low profile hand splint includes a section of a hollow sphere or ball that comfortably puts the hand in an at rest natural position, while immobilizing one or more anatomical hand parts with extended spherical sections thereby immobilizing fingers, knuckles and tendons. Straps hold the splint onto the palm and immobilize one or more of the fingers. An inverse version of the splint provides protection to the injured soft tissue of a palm.

A principal object and advantage of the present invention is that the splint isolates the hand and palm in a natural at rest position.

Another object and advantage is that the splint can be used for a myriad of injuries.

Another object and advantage of the present invention is that the splint is inexpensive and simple in its construction.

Another object and advantage of the present invention is that the splint comfortably supports the hand while permitting continued use of the hand and uninjured fingers.

Another object and advantage of the present invention is that donning clothes on and off is very simple as compared to the complicated prior art splints.

Another object and advantage is that the present splint may be used for initial injury care prior to the medical assessment as well as for healing support.

Another object and advantage is that the present splint is flexible to allow some hand/finger movement and is of a low profile in that it comfortably fits in the rest position palm of the hand as to not interfere with hand movements and operations.

Other objects and advantages will become apparent upon a reading of the following specification and claims and a review of the figures accompanying herewith.

### BRIEF DESCRIPTION OF THE DRAWINGS

FIG. 1 is a perspective view of a knuckle wrist immobilizing splint of the prior art.

FIG. 2 is a perspective view of the hand and palm wherein an injury or surgical area of the palm is covered by a thick bandage of the prior art.

FIG. 3 is a perspective view of the hand comfortably and naturally supported at rest by a sphere.

FIG. 4 is a similar view of FIG. 3 showing the anatomical parts of the hand comfortably resting on a sphere in phantom.

FIG. 5 is a perspective view of the present spherical section hand splint immobilizing a middle finger.

FIG. 6 is a perspective view of a second embodiment of the invention immobilizing two fingers of the hand.

FIG. 7 is a rear perspective view of the second embodiment of FIG. 6 showing the straps holding the splint in position.

FIG. 8 is a perspective view of the present spherical section hand splint with a textured rubber surface on the concave outer surface of the splint to aid the wearer in gripping objects such as a steering wheel.

FIG. 9 is a perspective view of a third embodiment of the present invention wherein the spherical section hand splint is inverted to protect the palm injury as shown in FIG. 2.

FIG. 10 is a perspective view of a fourth embodiment of the invention immobilizing three fingers of the hand.

FIGS. 11 and 12 are perspective view of the front and back of a fifth embodiment of the present invention in combination with a carpal tunnel brace.

### DETAILED SPECIFICATION

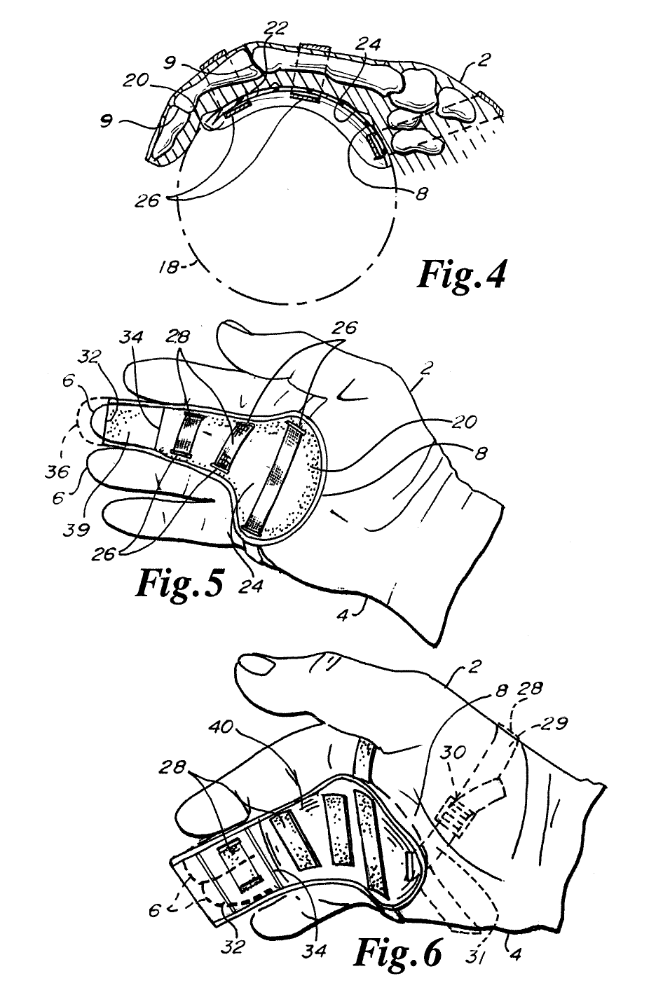

Referring to FIGS. 1 through 9, the hand 2 generally includes a wrist 4, fingers 6 and palm 8. The spherical hand splint 20 of the present invention may be viewed in FIGS. 4, 5, and 8. This splint 20 has its spherical section portion extending underneath a finger 6 to support and immobilize the finger 6 which further immobilizes injured or inflamed ligaments, tendons or knuckles 9 associated with injured fingers 6.

This spherical section hand splint 20 generally includes a concave palm surface 22 which fits up against a palm 8 of the hand 2. A convex exposed outer surface 24 faces outwardly from the palm 8. The splint 20 suitably has slots 26 therethrough to allow one end 29 of straps 28 with hook and loop surfaces to pass through slot 26, D-ring or buckle 30 of the other end 31 of the strap 28 and to lay back upon the strap 28 connecting a hook and loop surfaces. Alternatively, for short expanses, such and for securing fingers 6, the splint 20, the D-ring or buckle is not needed and the one end 29 simply lays upon strap 28 connecting hook and loop surfaces.

The splint 20 is suitable made of flexible plastic. Polypropylene works inexpensively well. One such plastic is called Natural PP Hornopolymer. Plastic allows the hand and fingers to flex the splint 20 when using the hand which offer some functionality during healing and is beneficial toward the end of the healing process before the splint 20 is removed.

VELCRO® ⅝ inch wide loop tape manufactured by Velcro USA Inc. of Manchester, N.H. works very well for straps 28. This manufacturer also makes VELSTRETCH® loop tape which works even better in certain applications requiring expansion and contraction of the strap 28. VELSTRETCH® is water resistant and provides about 55% stretch with a plush backing for comfort.

In FIG. 5, the splint 20 does have finger tip section 36 removed along score lines 32. This splint version 20 protects finger 6 but not the finger tip. This is because intermediate section 39 is still intacted with splint 20. The finger 6 cannot move as digits 9 (FIG. 4) are immobilized to splint 20 with straps 28.

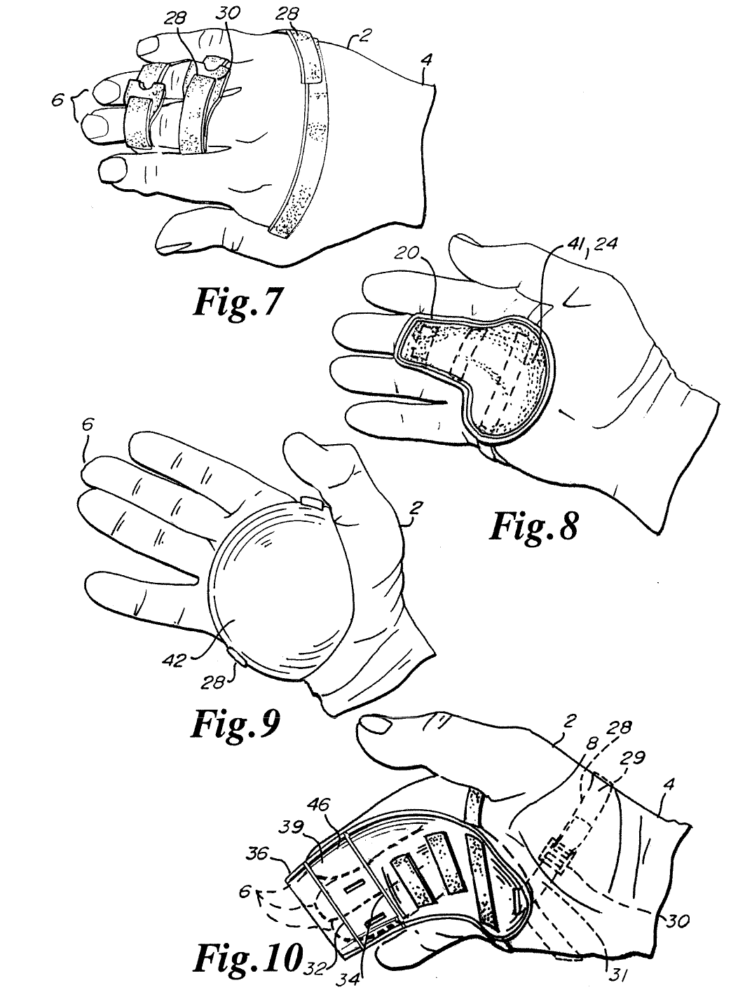

FIGS. 6 and 7 show a second embodiment of the spherical section hand splint 40 which has been tailored to support two fingers 6. It should readily be understood that the sphere portion of the hand splint 20 may adjustably be cut along score lines 32 and 34 to support as much of fingers 6 as necessary for rehabilitation to be accomplished. If knuckles are tender to touch or for some other reason, strap 28 around the wrist 4 may be used (FIG. 6).

In FIG. 6, the splint 40 does not have finger tip section 36 or intermediate section 39 removed along score lines 32 and 34. This splint version 40 completely protects fingers 6 beyond the finger tips. In FIG. 7, the finger tip section 36 and intermediate section 39 are removed along score line 34. As such, the two finger 6 tips may touch, feel and functionally move up to one or two digits 9 (FIG. 4).

FIG. 8 shows the present splint 20 with a textured rubber surface 41 on the convex outer surface 24 which will assist the wearer in grabbing objects, such as a steering wheel, to perform functions while isolating and immobilizing injured fingers, knuckles or tendons. The flexibility of splint 20 also assists in these activities. Splint 20 has tip section 36 and intermediate section 39 removed so that finger 6 tip may touch, feel and functionally move up to one or two digits 9.

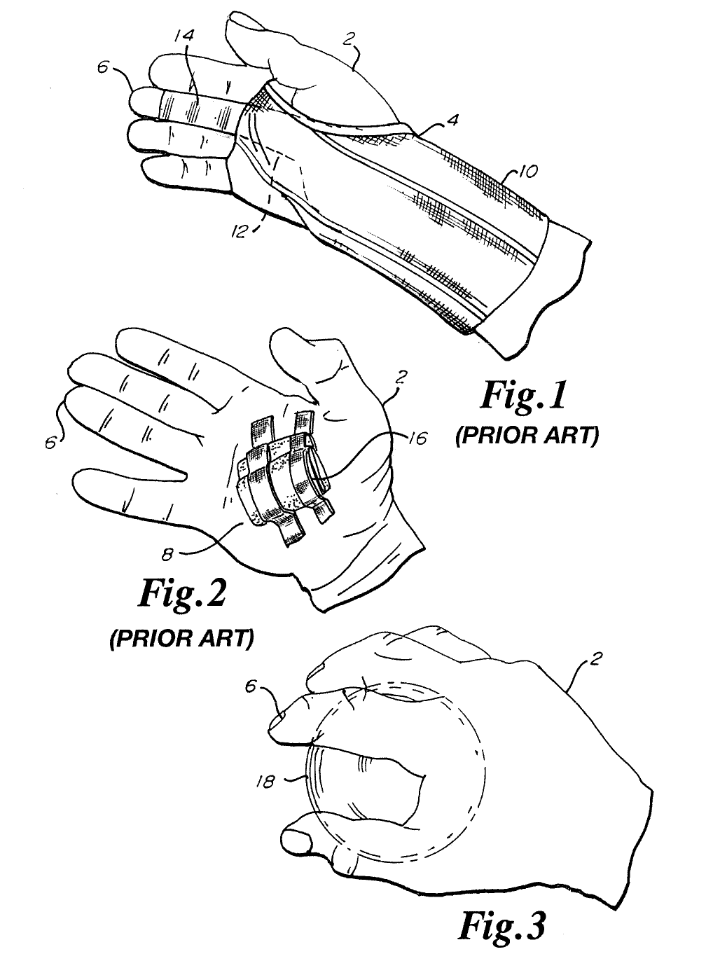

FIG. 9 shows a third embodiment 42 of the present invention which suitably covers the bandage 16 shown in FIG. 2 and thereby protects the injured palm from additional injury or bumpage which may cause additional medical problems and associated pain. Strap 28 also secures splint 42 to the palm 8.

FIG. 10 shows a fourth embodiment 46 of the spherical section hand splint which has been tailored to completely support three fingers 6. Again it should readily be understood that the spherical portion of the hand splint 46 may adjustably be cut to support much of fingers 6 for rehabilitation as desired. The finger tip section 36 and intermediate section 39 may be removed along score lines 32 or 34. Also if knuckles are tender to touch or for some other reason, strap 28 around the wrist 4 with buckle 30 may be used and one strap 28 adjacent the tender knuckle is eliminated, if needed.

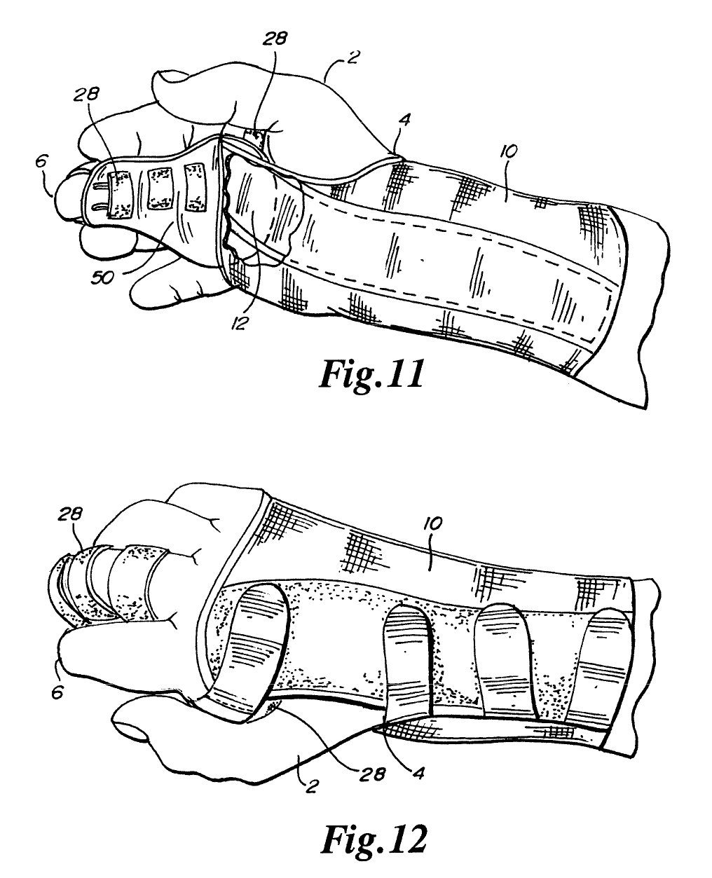

FIGS. 11 and 12 show a fifth embodiment 50 of the present invention in combination with carpal tunnel syndrome brace 10. Aluminum brace 12 within brace 10 overlaps a portion of splint 50 (in phantom outline) to hold splint 50 in place with the further assistance of dorsal strap 28.

The above embodiments are shown and described for illustrative purposes. The true scope of the present invention is intended to be covered by the following claims.

What is claimed is:

1. A flexible, low profile spherical hand splint that comfortably puts the hand in an at rest natural position immobilizing and supporting injured fingers, knuckles, tendons and palm portions, comprising:
a) a spherical section of a hollow sphere with a concave surface that comfortably fits into the palm of the hand; and
b) at least one strap that is connected to the spherical section for wrapping around a portion of the hand to hold and secure the splint onto the palm immobilizing and supporting injured fingers, knuckles, tendons and palm portions.

2. The hand splint of claim 1, further comprising a spherical section extension wherein the extension extends from the spherical section underneath at least one injured finger to naturally support the injured finger with least one additional strap that is connected to the extension for wrapping around a portion of the injured finger to hold and secure the injured finger to the splint.

3. The hand splint of claim 2, wherein the spherical section extension is wide enough to support and immobilize multiple injured fingers.

4. The hand splint of claim 2, wherein the spherical section extension has at least one transverse score line for shortening the extension to expose the injured finger tip.

5. The hand splint of claim 4, wherein the spherical section extension has a second transverse score line for shortening the extension to expose a knuckle of the injured finger tip.

6. The hand splint of claim 1, further comprising a peripheral slot in the spherical section for passing the strap therethrough to secure the splint to the palm.

7. The hand splint of claim 6, further comprising multiple peripheral slots in the spherical section for passing the straps therethrough to secure the splint to the palm and the injured fingers.

8. The hand splint of claim 1 wherein the splint is made from flexible plastic.

9. A flexible, low profile spherical hand splint that comfortably puts the hand in an at rest natural position immobilizing and supporting injured fingers, knuckles, tendons and palm portions, comprising:
a) a spherical section of a hollow sphere with a concave surface that comfortably fits into the palm of the hand;
b) at least one strap that is connected to the spherical section for wrapping around a portion of the hand to hold and secure the splint onto the palm immobilizing and supporting injured fingers, knuckles, tendons and palm portions; and
c) a spherical section extension wherein the extension extends from the spherical section underneath at least one injured finger to naturally support the injured finger with least one additional strap that is connected to the extension for wrapping around a portion of the injured finger to hold and secure the injured finger to the splint.

10. The hand splint of claim 9, wherein the spherical section extension is wide enough to support and immobilize multiple injured fingers.

11. The hand splint of claim 9, wherein the spherical section extension has at least one transverse score line for shortening the extension to expose the injured finger tip.

12. The hand splint of claim 11, wherein the spherical section extension has a second transverse score line for shortening the extension to expose a knuckle of the injured finger tip.

13. The hand splint of claim 9, further comprising a peripheral slot in the spherical section for passing the strap therethrough to secure the splint to the palm.

14. The hand splint of claim 13, further comprising multiple peripheral slots in the spherical section for passing the straps therethrough to secure the splint to the palm and the injured fingers.

15. The hand splint of claim 9 wherein the splint is made from flexible plastic.

16. A flexible plastic, low profile spherical hand splint that comfortably puts the hand in an at rest natural position immobilizing and supporting injured fingers, knuckles, tendons and palm portions, comprising:
a) spherical section of a hollow sphere with a concave surface that comfortably fits into the palm of the hand;
b) at least one strap that is connected to the spherical section for wrapping around a portion of the hand to hold and secure the splint onto the palm immobilizing and supporting injured fingers, knuckles, tendons and palm portions;
c) a spherical section extension wherein the extension extends from the spherical section underneath at least one injured finger to naturally support the injured finger with least one additional strap that is connected to the extension for wrapping around a portion of the injured finger to hold and secure the injured finger to the splint; and
d) wherein has at least one transverse score line on the spherical section extension for shortening the extension to expose a portion of the injured finger.

17. The hand splint of claim 16, wherein the spherical section extension is wide enough to support and immobilize multiple injured fingers.

18. The hand splint of claim 16, wherein the spherical section extension has a second transverse score line for shortening the extension to expose a knuckle of the injured finger tip.

19. The hand splint of claim 16, further comprising a peripheral slot in the spherical section for passing the strap therethrough to secure the splint to the palm.

20. The hand splint of claim 19, further comprising multiple peripheral slots in the spherical section for passing the straps therethrough to secure the splint to the palm and the injured fingers.
